(19) United States
(12) Patent Application Publication
(10) Pub. No.: US 2011/0022117 A1
(43) Pub. Date: Jan. 27, 2011

(54) Cytokine Production Via Electrical Stimulation to Treat Glaucoma

(76) Inventors: Casey Lind, Irvine, CA (US); Robert J. Sanchez, JR., Oceanside, CA (US)

Correspondence Address: ALCON, IP LEGAL, TB4-8, 6201 SOUTH FREEWAY, FORT WORTH, TX, 76134, US

(21) Appl. No.: 12/507,232

(22) Filed: Jul. 22, 2009

(51) Int. Cl. A61F 9/00; A61N 1/36

(52) U.S. Cl. 607/53

(57) ABSTRACT

The present invention is a method of treating glaucoma by electrically stimulating the ciliary muscle to produce shear stress on the trabecular meshwork sufficient for cytokine production. A device for electrically stimulating the ciliary muscle includes a pair of electrodes configured to provide a voltage to the ciliary muscle. The electrodes are coupled to a voltage source. A controller operates to apply the voltage to the ciliary muscle.

### BACKGROUND OF THE INVENTION

The present invention relates to a treatment for glaucoma and more particular to a method and apparatus for electrically stimulating the ciliary muscle of the eye to produce cytokines.

Glaucoma, a group of eye diseases affecting the retina and optic nerve, is one of the leading causes of blindness worldwide. Glaucoma results when the intraocular pressure (IOP) increases to pressures above normal for prolonged periods of time. IOP can increase due to an imbalance of the production of aqueous humor and the drainage of the aqueous humor. Left untreated, an elevated IOP causes irreversible damage to the optic nerve and retinal fibers resulting in a progressive, permanent loss of vision.

The eye's ciliary body epithelium constantly produces aqueous humor, the clear fluid that fills the anterior chamber of the eye (the space between the cornea and iris). The aqueous humor flows out of the anterior chamber through the trabecular meshwork, Schlemm's canal, and collector channels as well as the uveoscleral pathways, a complex drainage system. The delicate balance between the production and drainage of aqueous humor determines the eye's IOP.

Open angle (also called chronic open angle or primary open angle) is the most common type of glaucoma. With this type, even though the anterior structures of the eye appear normal, aqueous fluid builds within the anterior chamber, causing the IOP to become elevated. Left untreated, this may result in permanent damage of the optic nerve and retina. Eye drops are generally prescribed to lower the eye pressure. In some cases, surgery is performed if the IOP cannot be adequately controlled with medical therapy.

Only about 10% of the population suffers from acute angle closure glaucoma. Acute angle closure occurs because of an abnormality of the structures in the front of the eye. In most of these cases, the space between the iris and cornea is more narrow than normal, leaving a smaller channel for the aqueous humor to pass through. If the flow of aqueous humor becomes completely blocked, the IOP rises sharply, causing a sudden angle closure attack.

Secondary glaucoma occurs as a result of another disease or problem within the eye such as: inflammation, trauma, previous surgery, diabetes, tumor, and certain medications. For this type, both the glaucoma and the underlying problem must be treated.

FIG. 1 is a diagram of the front portion of an eye that helps to explain the processes of glaucoma. In FIG. 1, representations of the lens 110, cornea 120, iris 130, ciliary bodies 140, ciliary muscle 145, trabecular meshwork 150, and Schlemm's canal 160 are pictured. Anatomically, the anterior chamber of the eye includes the structures that cause glaucoma. Aqueous humor is produced by the ciliary bodies 140 that lie beneath the iris 130 and adjacent to the lens 110 in the anterior chamber. This aqueous humor washes over the lens 110 and iris 130 and flows to the drainage system located in the angle of the anterior chamber. The angle of the anterior chamber, which extends circumferentially around the eye, contains structures that allow the aqueous humor to drain. The first structure, and the one most commonly implicated in glaucoma, is the trabecular meshwork 150. The trabecular meshwork 150 extends circumferentially around the anterior chamber in the angle. The trabecular meshwork 150 seems to act as a filter, limiting the outflow of aqueous humor and providing a back pressure producing the IOP. Schlemm's canal 160 is located beyond the trabecular meshwork 150. Schlemm's canal 160 has collector channels that allow aqueous humor to flow out of the anterior chamber. The two arrows in the anterior chamber of FIG. 1 show the flow of aqueous humor from the ciliary bodies 140, over the lens 110, over the iris 130, through the trabecular meshwork 150, and into Schlemm's canal 160 and its collector channels.

The present invention provides a method of applying an electrical stimulus to the ciliary muscle so as to generate shear stress across the trabecular meshwork. This shear stress produces cytokines that increase the flow of aqueous humor through the trabecular meshwork. An implantable electrical stimulating device and external probe is also disclosed.

### SUMMARY OF THE INVENTION

In one embodiment consistent with the principles of the present invention, the present invention is a method of treating glaucoma by electrically stimulating the ciliary muscle to contract which in turn produces shear stress on the trabecular meshwork sufficient for cytokine production.

In another embodiment consistent with the principles of the present invention, the present invention is a method of treating glaucoma comprising providing a pair of electrodes coupled to a voltage source; placing the electrodes in, on or near the ciliary muscle; and applying a voltage across the pair of electrodes sufficient to stimulate the ciliary muscle to contract.

In another embodiment consistent with the principles of the present invention, the present invention is a device for electrically stimulating the ciliary muscle. The device has a pair of electrodes configured to provide a voltage to the ciliary muscle. The electrodes are coupled to a voltage source. A controller operates to apply the voltage to the ciliary muscle.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a diagram of the front portion of an eye.

FIG. 2 is a diagram of the front portion of the eye with the treatment areas highlighted to explain a method of treating the eye consistent with the principles of the present invention.

FIG. 3 is a block diagram of an electrical stimulation device according to the principles of the present invention.

FIG. 4 is a perspective view of an implantable electrical stimulation device according to the principles of the present invention.

FIG. 5 is a perspective view of an external electrical stimulation device according to the principles of the present invention.

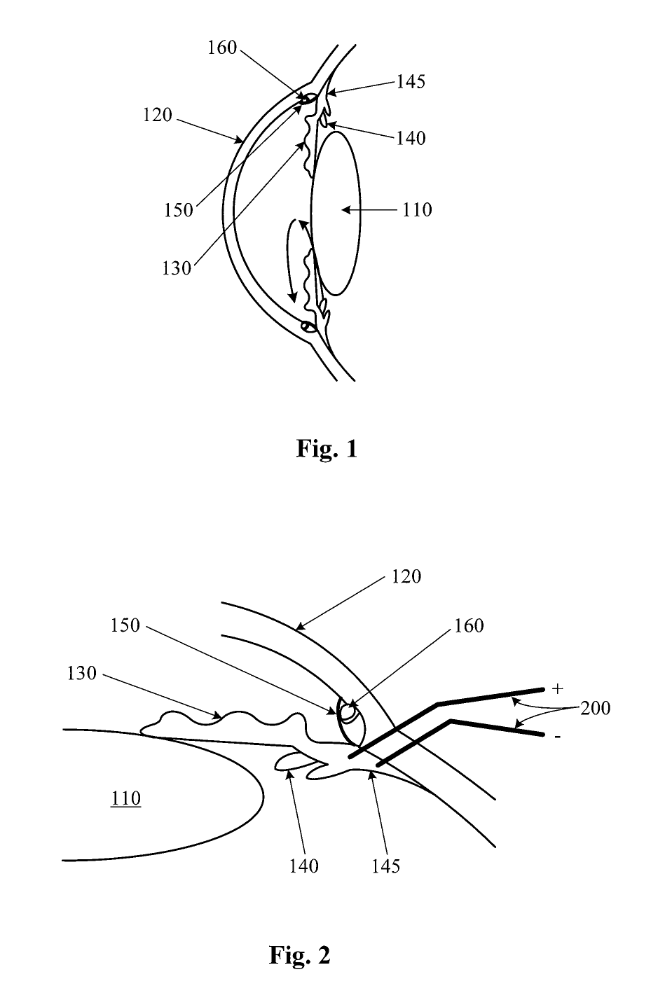

### DETAILED DESCRIPTION OF THE PREFERRED EMBODIMENTS

Reference is now made in detail to the exemplary embodiments of the invention, examples of which are illustrated in the accompanying drawings. Wherever possible, the same reference numbers are used throughout the drawings to refer to the same or like parts.

FIG. 2 is a diagram of the front portion of the eye with the treatment areas highlighted to explain a method of treating the eye consistent with the principles of the present invention. In FIG. 2, representations of the lens 110, cornea 120, iris 130, ciliary bodies 140, ciliary muscle 145, trabecular meshwork 150, and Schlemm's canal 160 are pictured. In addition, a pair of electrodes 200 is located at the treatment site. The pair of electrodes 200 is located in or on the ciliary muscle 145. A voltage applied across the electrodes 200 electrically stimulates the ciliary muscle 145. As the ciliary muscle 145 is stimulated, it relaxes and contracts. Since the ciliary muscle 145 is attached to the trabecular meshwork 150, movement of the ciliary muscle 145 produces a shear stress on the trabecular meshwork 150. This shear stress causes the trabecular meshwork 150 to generate cytokines. Cytokine production in the trabecular meshwork 150 leads to an increase in the flow of aqueous humor through the trabecular meshwork 150 and into Schlemm's canal 160 thus reducing IOP.

The electrodes would be used to deliver a direct current voltage that is sufficient to activate the ciliary muscle. The applied amperage may vary from 100 to 800 milliamperes.

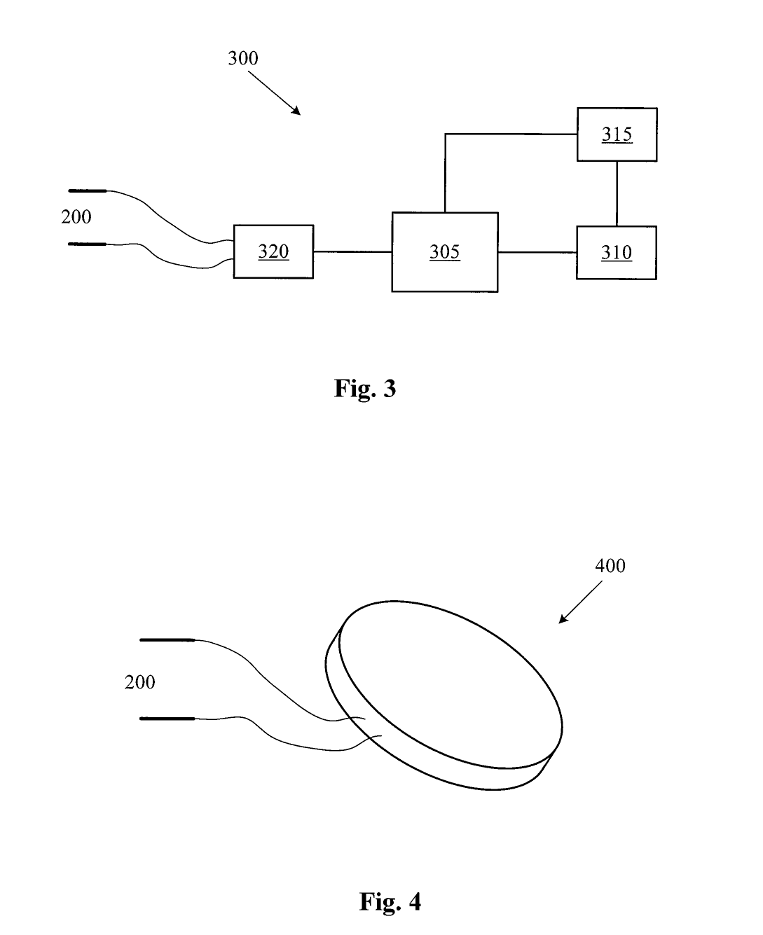

FIG. 3 is a block diagram of an electrical stimulation device according to the principles of the present invention. In FIG. 3, electrical stimulation device includes a controller 305, a voltage source 310, recharge circuitry 315, output circuitry 320, and a pair of electrodes 200.

Controller 305 is typically an integrated circuit with power, input, and output pins capable of performing logic functions. In various embodiments, processor 305 is a targeted device controller. In such a case, controller 305 performs specific control functions targeted to a specific device or component, such as a voltage source 310, recharge circuitry 315, or output circuitry 320. In other embodiments, controller 305 is a microprocessor. In such a case, controller 305 is programmable so that it can function to control more than one component of the device. In other cases, controller 305 is not a programmable microprocessor, but instead is a special purpose controller configured to control different components that perform different functions.

Voltage source 310 is typically a rechargeable battery, such as a lithium ion or lithium polymer battery, although other types of batteries may be employed. In addition, any other type of power cell is appropriate for voltage source 310. Voltage source 310 provides power to the device 300. Voltage source 310 can be recharged via an RFID link or other type of magnetic coupling via recharge circuitry 315.

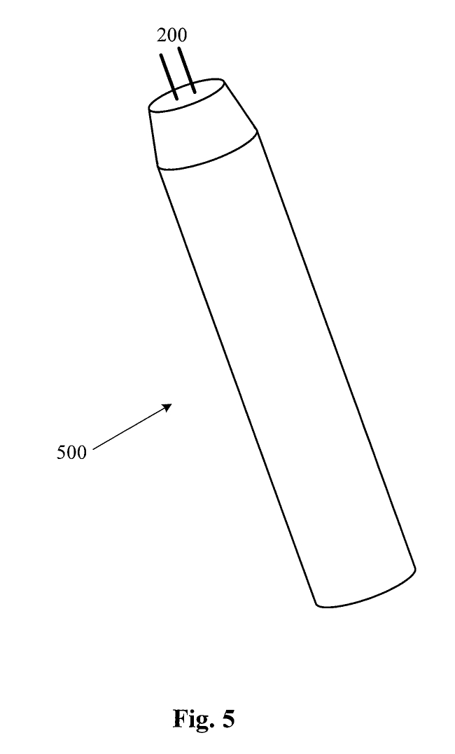

Recharge circuitry 315 charges voltage source 310. In one embodiment of the present invention, recharge circuitry 315 utilizes inductive coupling to charge voltage source 310. When device 300 is implanted in the eye, such an inductive charging technique is particularly useful. When device 300 is an external device (as in FIG. 5), voltage source 310 can be recharged in any of a number of conventional ways.3

Output circuitry 320 produces a voltage waveform that is applied across the pair of electrodes 200. Typically, output circuitry 320 produces pulses of voltage that are applied across the pair of electrodes 200. These voltage pulses stimulate the ciliary muscle.

This voltage will have a waveform associated with its delivery (sinusoidal, square or saw-tooth) that is matched to the required pattern needed to trigger the ciliary muscle.

The pair of electrodes 200 is sized so as to fit in or on the ciliary muscle. As such, the electrodes 200 are small, preferably on the scale of 10 to 200 micro meters in length and diameter whose cross-sectional shape maybe circular or of a polygon nature.

System 300 is preferably in a small, implantable, integrated package. As such, all of the components of system 300 can be built on a substrate, such as a semiconductor wafer, by any of a number of different processes.

FIG. 4 is a perspective view of an implantable electrical stimulation device according to the principles of the present invention. The device of FIG. 4 may contain some or all of the components described in the block diagram of FIG. 3. In FIG. 4, the implantable stimulation device 400 is generally disc-shaped and conforms to the curvature of the eyeball. A pair of electrodes 200 is connected to the implantable stimulation device 400 via lead wires. Typically, the implantable stimulation device 400 has a sealed enclosure that is suitable for implantation in the eye. As such, the implantable stimulation device 400 may have a stainless steel case as an outer shell. The implantable stimulation device 400 is inserted under the conjunctiva near the location of the ciliary muscle. In one case, the implantable stimulation device 400 may be inserted under the conjunctive near the limbus. Typically, the implantable stimulation device 400 is small—much less than the size of a dime.

FIG. 5 is a perspective view of an external electrical stimulation device according to the principles of the present invention. The device of FIG. 5 may contain some or all of the components described in the block diagram of FIG. 3. The stimulation device 500 has a pair of electrodes 200 that are sized and shaped to penetrate the sclera and contact the ciliary muscle. The stimulation device 500 is configured to be held in the hand and applied to the eye. In one embodiment, the pair of electrodes 200 has sharp distal ends that penetrate the sclera (at or near the limbus) and contact the ciliary muscle. In another aspect of the present invention, the pair of electrodes 200 does not penetrate the eye. Instead, the pair of electrodes 200 rests on the surface of the eye and transmits a voltage sufficient to stimulate the ciliary muscle.

From the above, it may be appreciated that the present invention provides a system treating glaucoma. The present invention provides an electrical stimulation device that applies a voltage to stimulate the ciliary muscle. Since the ciliary muscle is attached to the trabecular meshwork, movement of the ciliary muscle produces a shear stress on the trabecular meshwork. This shear stress causes the trabecular meshwork to generate cytokines. Cytokine production in the trabecular meshwork leads to an increase in the flow of aqueous humor through the trabecular meshwork and into Schlemm's canal thus reducing IOP. The present invention is illustrated herein by example, and various modifications may be made by a person of ordinary skill in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of treating glaucoma comprising:
electrically stimulating the ciliary muscle to produce shear stress on the trabecular meshwork sufficient for cytokine production.

2. A method of treating glaucoma comprising:
providing a pair of electrodes coupled to a voltage source;
placing the electrodes in, on or near the ciliary muscle;
applying a voltage across the pair of electrodes sufficient to stimulate the ciliary muscle.

3. The method of claim 2 wherein the voltage applied to the ciliary muscle produces stress in the trabecular meshwork sufficient to generate cytokines.

4. The method of claim 2 wherein placing the electrodes on or near the ciliary muscle further comprises:
implanting the electrodes under the sclera.

5. The method of claim 2 wherein placing the electrodes on or near the ciliary muscle further comprises:
implanting the electrodes under the conjunctiva.

6. The method of claim 2 wherein placing the electrodes on or near the ciliary muscle further comprises:
implanting the electrodes in the ciliary muscle.

7. The method of claim 2 wherein the voltage applied is a series of voltage pulses.

8. A device for electrically stimulating the ciliary muscle, the device comprising:
a pair of electrodes configured to provide a voltage to the ciliary muscle;
a voltage source coupled to the pair of electrodes; and
a controller operable to apply the voltage to the ciliary muscle.

9. The device of claim 8 further comprising:
recharge circuitry for recharging the voltage source.

10. The device of claim 8 further comprising:
output circuitry for producing a voltage that is applied to the electrodes.

11. The device of claim 8 further comprising:
a shell enclosing the voltage source and controller, the shell suitable for implantation into the eye.

12. The device of claim 8 wherein the pair of electrodes have sharp distal ends capable of puncturing the eye.
